(19) United States
(12) Patent Application Publication
(10) Pub. No.: US 2013/0095535 A1
(43) Pub. Date: Apr. 18, 2013

(54) ENZYMATIC RESOLUTION OF RACEMIC (2R,S)-2-(ACETYLAMINO)-3-METHOXY-N-(PHENYLMETHYL)PROPANAMIDE

(71) Applicants: Jordi Bosch i Lladó, Girona (ES); Ernesto Durán López, Castellbisbal (ES)

(72) Inventors: Jordi Bosch i Lladó, Girona (ES); Ernesto Durán López, Castellbisbal (ES)

(73) Assignee: MEDICHEM, S.A., Barcelona (ES)

(21) Appl. No.: 13/704,426

(22) PCT Filed: Jun. 15, 2011
(86) PCT No.: PCT/IB2011/052607

§ 371 Date: Dec. 14, 2012

Related U.S. Application Data
(60) Provisional application No. 61/434,149, filed on Jan. 19, 2011.
(60) Provisional application No. 61/354,961, filed on Jun. 15, 2010.

(51) Int. Cl. C12P 41/00; C12P 13/02

(52) U.S. Cl. CPC C12P 41/006; C12P 13/02
USPC 435/129; 435/280

(57) ABSTRACT

The present invention is concerned with a process of preparing (R)-lacosamide. The process comprises providing an (R,S)-lacosamide precursor and contacting the same with at least an enzyme in the presence of a solvent. The enzyme either stereoselectively hydrolyzes or acetylates an (R)- or (S)-enantiomer of the (R,S)-lacosamide precursor. The process further comprises where appropriate also concurrently, or successively, employing one or more reagents capable of converting the hydrolysed or acetylated (R)- or (S)-enantiomer to (R)-lacosamide.

### FIELD OF THE INVENTION

The present invention relates to processes for preparing (2R)-2-(acetylamino)-3-methoxy-N-(phenylmethyl)propanamide (i.e. lacosamide)

### BACKGROUND ART

Lacosamide (compound I) is the international commonly accepted name for (2R)-2-(acetylamino)-3-methoxy-N-(phenylmethyl)propanamide (also known as (R)-N-benzyl-2-acetamido-3-methoxypropionamide) and has an empirical formula of C₁₃H₁₈N₂O₃ and a molecular weight of 250.30 g/mol.

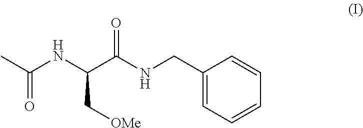 [SMILES: COC[C@@H](NC(C)=O)C(=O)NCc1ccccc1]

Lacosamide is an active substance indicated for adjunctive treatment of partial-onset seizures and diabetic neuropathic pain. In the United States, lacosamide is marketed under the trademark VIMPAT™ for the treatment of epilepsy.

The synthesis of lacosamide was first described in U.S. Pat. No. 5,773,475 (“the '475 patent”). In the '475 patent, lacosamide is prepared starting from D-Serine ((R)-2-Amino-3-hydroxypropionic acid), a chiral building block that has the desired stereochemistry, using three different approaches as disclosed in the following Schemes 1, 2, and 3.

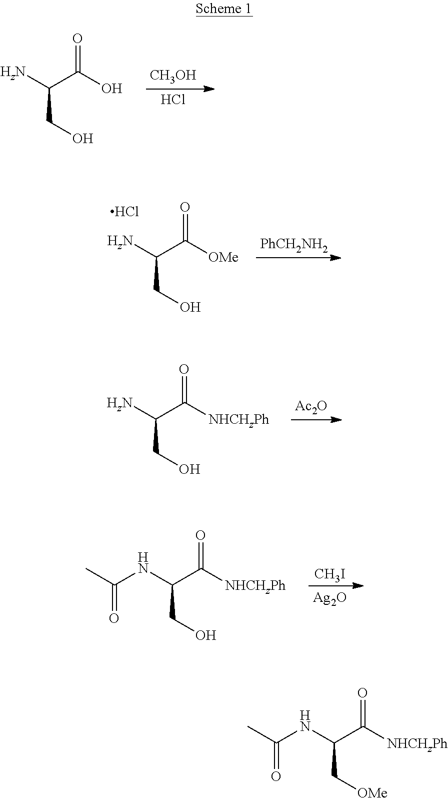

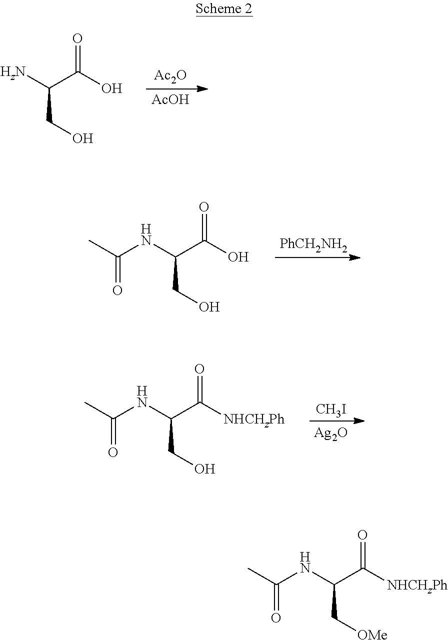

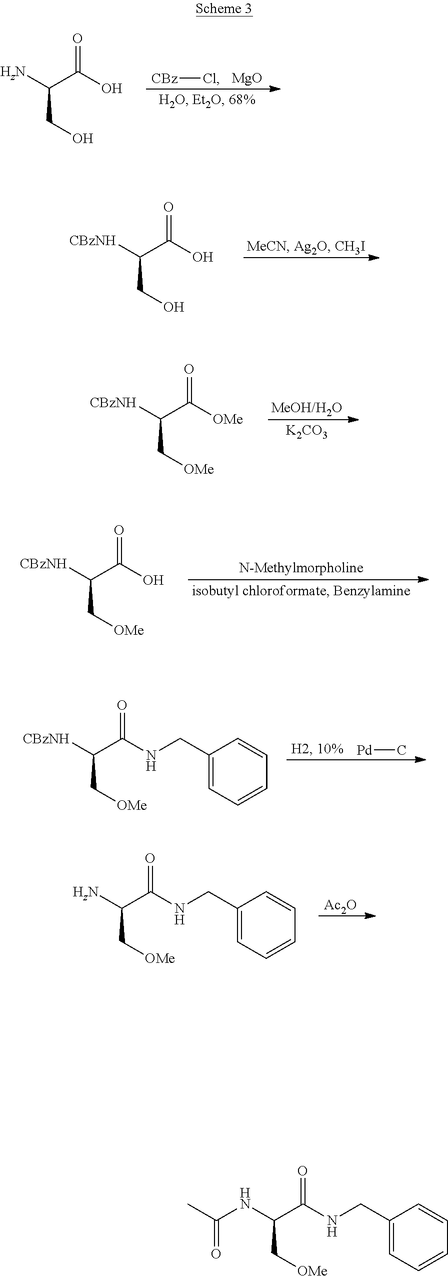

U.S. Pat. No. 6,048,899 (“the '899 patent”), a continuation-in-part of the '475 patent, includes an example wherein lacosamide is prepared starting from D-Serine, using a different approach as disclosed in the following Scheme 4.

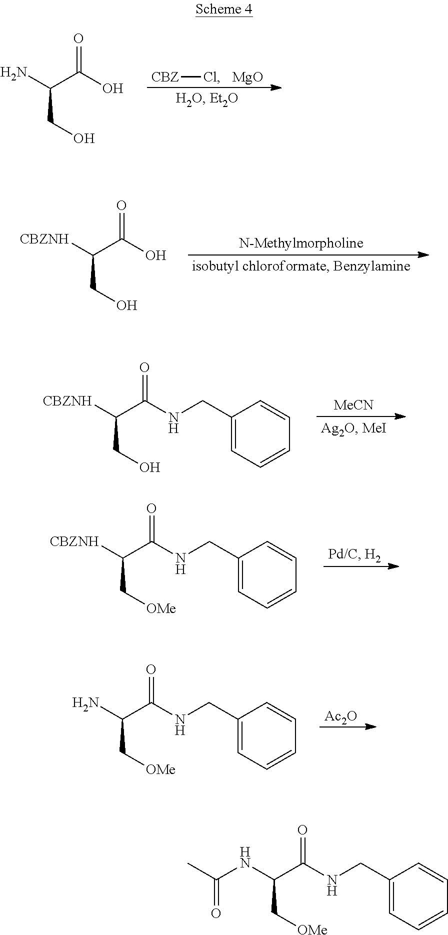

An improved process is described in U.S. Patent Application Publication No. 2008/0027137. In this application, the methylation step is carried out on N-Boc-D-Serine using dimethylsulphate and either n-butyl lithium or aqueous sodium hydroxide and phase transfer catalysis as disclosed in the following Scheme 5.

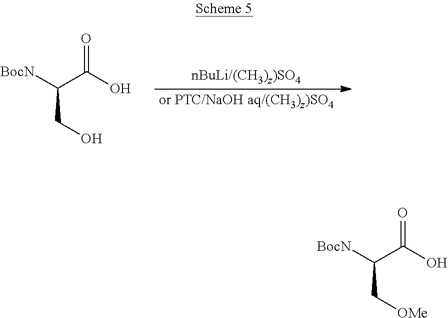

The resulting methoxy compound is transformed to lacosamide using similar reaction conditions as those depicted in Schemes 1 to 4.

Another improved process is described in the U.S. Patent Application Publication No. 2009/0143472. In this application, N-trityl-D-Serine is used as a starting material in order to minimize racemization due to the use of the bulky trityl protecting group.

However, syntheses of lacosamide described previously suffer from at least one of the following drawbacks: the use of methylating agents that are highly toxic and may lead to safety or environmental issues when producing lacosamide on a large scale, the use of expensive, unnatural D-Serine as starting building block and/or the tendency to racemization during the methylation step.

Some drawbacks of previously known lacosamide syntheses have been addressed in the International Patent Application WO 2010/052011. In this application, lacosamide is prepared starting from racemic relatively inexpensive raw materials. However, the final separation is carried out using chromatographic techniques such as Simulated Moving Bed. Although this is a well established technique, it requires significant capital investment, the recovery of high amounts of solvent by distillation and thus a relatively high operational expenditure. Furthermore, in WO 2010/052011 lacosamide is prepared by resolution of the racemic intermediate 2-amino-N-benzyl-3-methoxypropionamide by diastereomeric salt formation followed by acetylation of (R)-2-amino-N-benzyl-3-methoxypropionamide. Resolution by diastereomeric salt formation requires an adequate chiral resolving agent available in an optically pure form which is normally expensive.

Thus, there remains a need for an improved process for preparing lacosamide.

### BRIEF SUMMARY OF THE INVENTION

The invention provides a process for preparing (R)-lacosamide, which process comprises:

(i) providing an (R,S)-compound of formula (II)

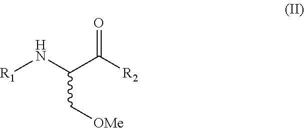

wherein:
R₁ represents either CH₃C(═O)—, or a first intermediate moiety that can be converted into CH₃C(═O)—; and
R₂ represents either —NHCH₂Ph, or a second intermediate moiety that can be converted into —NHCH₂Ph;

(ii) contacting the (R,S)-compound of formula (II) with at least an enzyme in the presence of a solvent, wherein the enzyme is selected such that either:
(a) when R₁ represents CH₃C(═O)— or said first intermediate moiety in an (R,S)-compound of formula (II) and in the presence of a protic solvent, said enzyme stereoselectively hydrolyzes either the (R)- or (S)-enantiomer thereof; or
(b) when R₁ represents said first intermediate moiety in an (R,S)-compound of formula (II) and in the presence of a reagent which is an acetyl donor compound, said enzyme stereoselectively acetylates the (R)- or (S)-enantiomer thereof;

whereby either (a) or (b) selectively results in enantiomerically enriched, or enantiomerically pure, (R)-enantiomer of the compound formula (II);

and where appropriate also concurrently, or successively, employing one or more reagents capable of converting said first intermediate moiety into CH₃C(═O)—, and/or said second intermediate moiety into —NHCH₂Ph.

### BRIEF DESCRIPTION OF THE DRAWINGS

FIG. 1 shows HPLC chromatogram of racemic 2-acetylamino 3-methoxypropanoic acid obtained in Example 1.

FIG. 2 shows HPLC chromatogram of (R)-2-acetylamino 3-methoxypropanoic acid obtained in Example 2.

FIG. 3 shows HPLC chromatogram of the crude reaction mixture obtained after the enzymatic reaction of example 3.

### DETAILED DESCRIPTION OF THE INVENTION

According to the present invention, therefore, there is provided a process of preparing (R)-lacosamide, which is (2R)-2-(acetylamino)-3-methoxy-N-(phenylmethyl)propanamide, of formula (I)

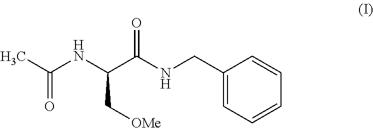 [SMILES: COC[C@@H](NC(C)=O)C(=O)NCc1ccccc1]

which process comprises:

(i) providing an (R,S)-compound of formula (II)

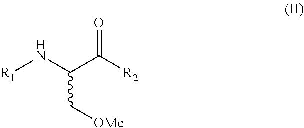

wherein:
R₁ represents either CH₃C(═O)—, or a first intermediate moiety that can be converted into CH₃C(═O)—; and
R₂ represents either —NHCH₂Ph, or a second intermediate moiety that can be converted into —NHCH₂Ph;

(ii) contacting the (R,S)-compound of formula (II) with at least an enzyme in the presence of a solvent, wherein the enzyme is selected such that either:
(a) when R₁ represents CH₃C(═O)— or said first intermediate moiety in an (R,S)-compound of formula (II) and in the presence of a protic solvent, said enzyme stereoselectively hydrolyzes either the (R)- or (S)-enantiomer thereof; or
(b) when R₁ represents said first intermediate moiety in an (R,S)-compound of formula (II) and in the presence of a reagent which is an acetyl donor compound, said enzyme stereoselectively acetylates the (R)- or (S)-enantiomer thereof;

whereby either (a) or (b) selectively results in enantiomerically enriched, or enantiomerically pure, (R)-enantiomer of the compound formula (II);

and where appropriate also concurrently, or successively, employing one or more reagents capable of converting said first intermediate moiety into CH₃C(═O)—, and/or said second intermediate moiety into —NHCH₂Ph.

Stereoselective hydrolysis according to the present invention can be carried out on either the (R) or (S)-enantiomer of formula (II) thereby providing (R)-lacosamide, or a desired (R)-enantiomer for subsequent reaction to yield (R)-lacosamide. For example, stereoselective hydrolysis of the (R)-enantiomer of formula (II) as above could result in a corresponding (R)-hydrolysis product that could typically then be separated by means of an acid wash, and subsequently transformed into (R)-lacosamide. Preferably, the stereoselective acetylation is, however, carried out on the (R)-enantiomer of formula (II), although it is also envisaged as above that the (S)-enantiomer could alternatively be employed followed by separation and process steps to yield (R)-lacosamide. For example, stereoselective actylation of the (S)-enantiomer of formula (II) as above could result in a corresponding (S)-acetylated product that could typically then be removed by means of extraction, and the non acetylated (R) enantiomer of formula (II) could then be transformed into (R)-lacosamide.

According to a preferred embodiment, there is thus provided a process of preparing (R)-lacosamide, which is (2R)-2-(acetylamino)-3-methoxy-N-(phenylmethyl)propanamide, of formula (I)

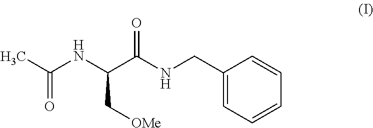 [SMILES: COC[C@@H](NC(C)=O)C(=O)NCc1ccccc1]

which process comprises:

(i) providing an (R,S)-compound of formula (II)

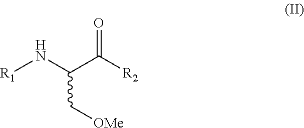

wherein:
R₁ represents either CH₃C(═O)—, or a first intermediate moiety that can be converted into CH₃C(═O)—; and
R₂ represents either —NHCH₂Ph, or a second intermediate moiety that can be converted into —NHCH₂Ph;

(ii) contacting the (R,S)-compound of formula (II) with at least an enzyme in the presence of a solvent, wherein the enzyme is selected such that either:
(a) when R₁ represents CH₃C(═O)— or said first intermediate moiety in an (R,S)-compound of formula (II) and in the presence of a protic solvent, said enzyme stereoselectively hydrolyzes the (R) or (S)-enantiomer thereof; or
(b) when R₁ represents said first intermediate moiety in an (R,S)-compound of formula (II) and in the presence of a reagent which is an acetyl donor compound, said enzyme stereoselectively acetylates the (R)-enantiomer thereof;

whereby either (a) or (b) selectively results in enantiomerically enriched, or enantiomerically pure, (R)-enantiomer of the compound formula (II) having R₁ representing CH₃C(═O)—;

and where appropriate also concurrently, or successively, employing a reagent capable of converting said second intermediate moiety into —NHCH₂Ph.

With respect to the stereoselective acetylation of a compound of formula (II) as described above, it is further preferred that the compound of formula (II) represents an (R,S)-compound of formula (IIa)

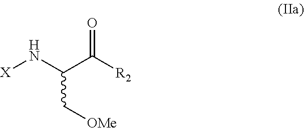

wherein R₂ is as defined above; and X represents H (preferred) or a typical leaving group; and as above the process comprises contacting the (R,S)-compound with at least a reagent which is an acetyl donor, and at least an enzyme that stereoselectively acetylates the (R)-enantiomer of said compound of formula (IIa), in the presence of a solvent, which selectively results in enantiomerically enriched, or enantiomerically pure (R)-enantiomer of formula (III)

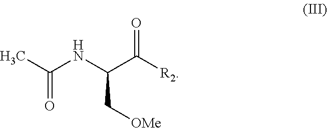

and where appropriate also concurrently, or successively, employing a reagent capable of converting said intermediate moiety to —NHCH₂Ph.

As indicated above, a preferred compound (IIa) is (2R,S)-2-amino-3-methoxy-N-(phenylmethyl)propanamide

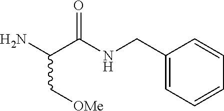 [SMILES: COCC(N)C(=O)NCc1ccccc1]

and as such stereoselectively acetylating the (R)-enantiomer of (2R)-2-amino-3-methoxy-N-(phenylmethyl)propanamide results in an intermediate mixture of enantiomerically enriched, or enantiomerically pure, (R)-lacosamide, together with enantiomerically enriched (2S)-2-amino-3-methoxy-N-(phenylmethyl)propanamide. What is meant herein by “enantiomerically enriched” (2S)-2-amino-3-methoxy-N-(phenylmethyl)propanamide is hereinbefore described. Typically, the process further comprises isolating enantiomerically enriched, or enantiomerically pure, (R)-lacosamide, from the intermediate mixture.

Suitable enzymes that can stereoselectively acetylate a compound of formula (II) or (IIa) are lipase enzymes. Preferably, the lipase is Candida antarctica lipase A (CAL-A), Candida antarctica lipase B (CAL-B), or Pseudomonas cepacia lipase, and especially preferred is Candida antarctica lipase B (CAL-B).

An acetyl donor suitable for use in the stereoselective acetylation of the present invention can be selected from the group consisting of acetic acid, acetate esters, acetyl-coenzyme A and acetamides. Preferably, the acetyl donor is a lower alkyl acetate ester, and preferably is ethyl acetate or isopropyl acetate.

The solvent for the stereoselective acetylation of the present invention is typically selected from the group consisting of water, organic solvents, and mixtures thereof. Preferably the solvent is an organic solvent, and as such can be selected from the group consisting of hydrocarbon solvents, ketone solvents, ether solvents, and ester and/or amide solvents containing an acyl moiety which is not acetyl. In a particularly preferred embodiment, the solvent is an ether solvent, preferably methyl tert-butyl ether. In an alternative particularly preferred embodiment, the solvent is a hydrocarbon solvent, and preferably is toluene.

Suitably, the stereoselective acetylation of the present invention is carried out at a temperature in the range of about 15 to 110° C., preferably in the range of about 20 to 50° C.

Furthermore, in certain embodiments of the stereoselective acetylation of the present invention the process is carried out in the presence of catalyst, in particular a ruthenium complex catalyst, such as Shvo's catalyst [i.e. 1-hydroxytetraphenylcyclopentadienyl(tetraphenyl-2,4-cyclopentadiene-1-one)-μ-hydrotetracarbonyldiruthenium (II)] and/or a palladium catalyst such as palladium in aluminium oxyhydroxide.

A preferred stereoselective acetylation according to the present invention can be represented by the following Scheme.

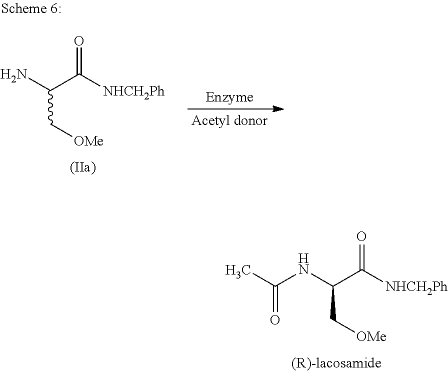

With respect to the stereoselective hydrolysis of a compound of formula (II) as described above according to the present invention, it is further preferred that a process according to the present invention comprises:

(i) providing an (R,S)-compound of formula (IIb)

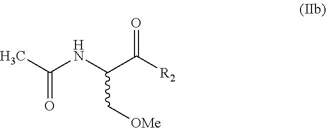

wherein:

R₂ represents either —NHCH₂Ph, or an intermediate moiety that can be converted into —NHCH₂Ph;

(ii) contacting the (R,S)-compound of formula (IIb) with at least an enzyme that stereoselectively hydrolyzes the (R) or (S)-enantiomer of said compound of formula (IIb), in the presence of a protic solvent, so as to selectively result in enantiomerically enriched, or enantiomerically pure (R)-enantiomer of formula (III)

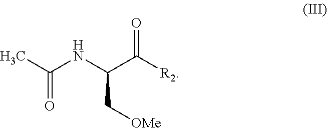

and where appropriate also concurrently, or successively, employing a reagent capable of converting said intermediate moiety to —NHCH₂Ph.

In a preferred embodiment of a hydrolysis process according to the present invention, the enzyme hydrolyzes the (S)-enantiomer of an (R,S)-compound of formula (IIb). According to this embodiment of the present invention, compound (IIb) can be (2R,S)-2-(acetylamino)-3-methoxy-N-(phenylmethyl)propanamide

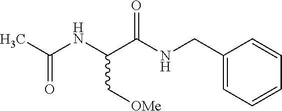 [SMILES: COCC(NC(C)=O)C(=O)NCc1ccccc1]

in other words (R,S)-lacosamide, whereby the enzyme stereoselectively hydrolyzes the (S)-enantiomer of lacosamide, preferably the acetylamino group thereof, so as to selectively result in enantiomerically enriched, or enantiomerically pure (R)-lacosamide.

The above stereoselective hydrolysis of (R,S)-lacosamide can result in a mixture comprising desacetyl-(S)-lacosamide (i.e., compound (V) below in Scheme 7) and the unreacted (R) enantiomer of lacosamide. The (R,S)-lacosamide can be either a racemic mixture or an enantiomerically enriched mixture substantially as hereinbefore described. Preferably, racemic lacosamide is used as the (R,S)-lacosamide starting material. Racemic lacosamide can be prepared by any of the methods described in the International Patent Application WO 2010/052011 or by epimerization of undesired (S)-lacosamide or (R)-lacosamide with a low enantiomeric excess, under basic conditions.

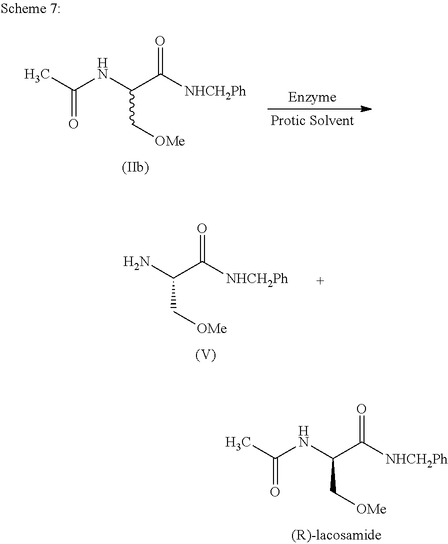

Preferably, racemic lacosamide is dissolved or suspended in a protic solvent (e.g., water) in an amount to obtain a concentration of about 0.05 to 1 mol per litre. The pH is then preferably adjusted to about 4 to about 9 by the addition of the required amounts of a suitable acid or base. A buffering agent also may be used. After the addition of a suitable amount of the enzyme, the reaction progress can be monitored by a suitable analytical method, preferably a chiral HPLC method capable of separating the 2 enantiomers of lacosamide, or alternatively by a colorimetric ninhydrine based method capable of monitoring the presence of free (non acylated) derivative (i.e. compound V). Once the reaction is complete, (R)-lacosamide is advantageously extracted for example by using a solvent not miscible with water, and purified by conventional methods known in the art such as extraction and/or crystallization. Optionally, before performing the extraction of (R)-lacosamide, either an enzyme immobilized on a solid support can be removed from the reaction mixture for example by filtration and/or the reaction mixture can be acidified using a suitable acid, for example hydrochloric acid or any other mineral acid, for a better removal of the undesired desacetyl-(S)-lacosamide (compound V) and the enzyme with the aqueous phase. Also, the undesired desacetyl-(S)-lacosamide (compound V) can be precipitated by formation of a suitable acid addition salt and removed by filtration.

In an alternative preferred embodiment of a hydrolysis process according to the present invention, where the enzyme hydrolyzes the (S)-enantiomer of an (R,S)-compound of formula (IIb), compound (IIb) is (2R,S)-2-(acetylamino)-3-methoxypropionic acid

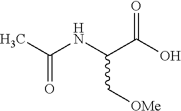

whereby said enzyme stereoselectively hydrolyzes the (S)-enantiomer thereof, so as to obtain a mixture comprising enantiomerically enriched, or enantiomerically pure (R)-intermediate (IIIa) and hydrolysis product (IV)

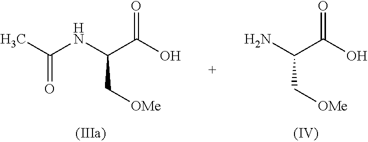

and converting (R)-intermediate (IIIa) into enantiomerically enriched, or enantiomerically pure (R)-lacosamide.

The above preferred embodiment according to the present invention can be further illustrated by Scheme 8 below.

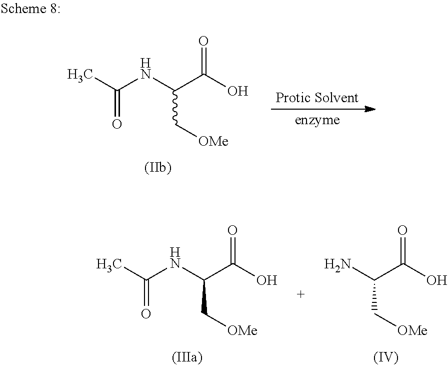

Preferably, racemic compound (IIb) is dissolved or suspended in a protic solvent, still preferably in water, in an amount to obtain a concentration of about 0.05 to 1 mol per litre, preferably about 0.1 to 0.5 mol per litre, still more preferably about 0.2 mol per litre. According to a preferred aspect of the invention, the pH is then adjusted to about 4 to about 9, preferably to about 7, by the addition of an adequa base such as an alkaline or alkaline earth hydroxide, and preferably lithium, sodium or potassium hydroxide. A buffering agent also may be used, preferably a neutral phosphate buffer (pH=7) is used. After the addition of a suitable amount of the enzyme, i.e. about 1 to 10,000 units of enzyme per g of compound (IIb), preferably about 10 to 5,000 units of enzyme per g of compound (IIb), still more preferably about 100 to 2,000 units of enzyme per g of compound (IIb), even more preferably about 500 to 1,000 units of enzyme per g of compound (IIb), the reaction mixture is preferably heated to about 25° C. to about 50° C., preferably to about 37° C. to about 40° C., and stirred at this temperature until the completion of the reaction. The reaction progress can be typically monitored by a suitable analytical method, preferably a chiral HPLC method capable of separating the 2 enantiomers of compound (IIb), or alternatively by a colorimetric ninhydrine based method capable of monitoring the presence of the free (non acylated) amino acid derivative. Once the reaction is complete, compound (IIIa) is preferably extracted for example by using a solvent not miscible with water, and purified by conventional methods known in the art. Optionally, before performing the extraction of compound (IIIa), either an enzyme immobilized on a solid support can be removed from the reaction mixture for example by filtration and/or the reaction mixture can be acidified using a suitable acid, for example hydrochloric acid or any other mineral acid, for a better removal of the undesired free (non acylated) amino acid derivative of the (S) enantiomer of compound (IV) and the enzyme with the aqueous phase. Also, the undesired free (non acylated) amino acid derivative of the (S) enantiomer of compound (IV) can be precipitated by formation of a suitable acid addition salt and removed by filtration.

Illustrative suitable enzymes suitable for use in stereoselective hydrolysis the of the (S)-enantiomer of an (R,S)-compound of formula (IIb) according to the present invention include Acylase I (also known as aminoacylase I or N-acylamino-acid amidohydrolase, EC 3.5.1.14) or other enzymes with an enhanced activity or enantioselectivity for this reaction.

In a alternative embodiment of a hydrolysis process according to the present invention, the enzyme hydrolyzes the (R)-enantiomer of an (R,S)-compound of formula (IIb). In this embodiment an (R,S)-compound of formula (IIb) can be

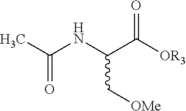

wherein R₃ is as defined below, and preferably is C₁ to C₆ alkyl,

whereby said enzyme stereoselectively hydrolyzes the (R)-enantiomer thereof, preferably the ester group thereof, so as to obtain enantiomerically pure (R)-intermediate (IIIa) (2R)-2-(acetylamino)-3-methoxypropionic acid

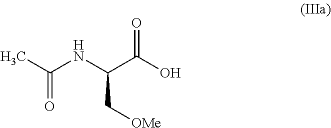

and converting (R)-intermediate (IIIa) into enantiomerically enriched, or enantiomerically pure (R)-lacosamide.

The above preferred embodiment according to the present invention can be further illustrated by Scheme 9 below.

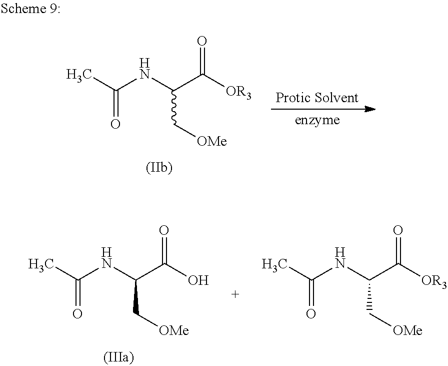

In this embodiment, compound (Ma) is obtained by stereoselective hydrolysis of compound (IIb) using a suitable enzyme, thus obtaining a mixture comprising the unreacted (S) enantiomer of compound (IIb) and the free (non esterified) carboxylic acid derivative of the (R) enantiomer of compound (IIb) (i.e., compound (IIIa)) in the reaction mixture (see Scheme 9 above), wherein compound (IIb) can be either a racemic mixture or an enantiomerically enriched mixture. Preferably, racemic compound (IIb) is used as starting material. Illustrative suitable enzymes include a lipase or an esterase or other enzymes with an enhanced activity or enantioselectivity for this reaction.

Compound (IIb) can be obtained by esterification of the corresponding carboxylic acid under any conventional method described in the art, wherein R₃ in compound (IIb) as above can be selected from the group comprising alkyl, alkenyl, alkynyl, cycloalkyl, cycloalkenyl, cycloalkynyl, aralkyl, heteroaralkyl, aryl or heteroaryl, which groups may optionally be mono- or polysubstituted; and wherein each ring independently may optionally be condensed with one or more homo- or heterocyclic rings, and one or more carbon atoms of the saturated and unsaturated rings may optionally be replaced with one or more heteroatoms selected from nitrogen, oxygen and sulphur atom. Preferably, R is C₁ to C₆ alkyl as referred to above, still preferably R is C₁ to C₃ alkyl, and more preferably is methyl or ethyl.

Preferably, racemic compound (1%) is dissolved or suspended in a protic solvent (e.g., water) in an amount to obtain a concentration of about 0.05 to 1 mol per litre. According to a preferred aspect of the invention, the pH is then adjusted to about 4 to about 9 by the addition of a suitable acid or base. A buffering agent also may be used. After the addition of a suitable amount of the enzyme, the reaction progress can be monitored by a suitable analytical method, preferably a chiral HPLC method capable of separating the 2 enantiomers of compound (IIb). Advantageously, once the reaction is complete, the reaction mixture is basified using a suitable base, for example lithium, sodium or potassium hydroxide or any other inorganic or organic base, and the undesired (S) enantiomer of compound (IIb) is preferably removed by extraction for example by using a solvent not miscible with water. Finally, the aqueous phase can be acidified using an adequate acid, for example hydrochloric acid or any other mineral acid, and compound (Ma) can be then extracted using a solvent not miscible with water, and purified by conventional methods known in the art. Optionally, compound (IIIa) can be isolated from the reaction mixture by precipitation of a salt of compound (Ma) using a suitable base and removing this salt of compound (Ma) by filtration.

Enzymes as used in either the stereoselective acetylation or hydrolysis of the present invention may be naturally occurring enzyme, or a synthetic enzyme obtained by genetic modification. The enzymes may thus be used in crude form or as purified extract, which may be soluble in at least the solvent of the reaction mixture, or may be insoluble such as immobilized on a solid support. Typically, the enzyme is present in an amount in the range of 1 to 10,000 units of enzyme per gram of (R,S)-compound (II), (IIa) or (IIb) as described herein.

A process according to the present invention can further preferably comprise converting a compound of formula (III) or (IIIa) as described herein into enantiomerically enriched, or enantiomerically pure (R)-lacosamide, comprises O-benzylaminating using an O-amination activating agent and an O-benzylaminating agent. Suitably, the O-amination activating agent is 1,1′-carbonyldiimidazole (CDI), dicyclohexylcarbodiimide (DCC), or T3P™ and the O-benzylaminating agent is benzylamine.

A process according to the present invention can further comprise a non enzymatic acetylation by means of the use of a standard acetylation agent. The acetylation agent could be an acetyl donor substantially as hereinbefore described, or could be a different acetylation agent such as acetic anhydride or an acetic halide such as acetic chloride.

It is still further preferred that a process of the present invention involves monitoring the extent of the respective enzymic reaction, for example respectively detecting unreacted (R,S)-compound (II), (IIa) or (IIb), and/or as appropriate unreacted hydrolysis product (IV).

The processes of the present invention, under optimized conditions, may afford both enantiomers of lacosamide in high enantiomeric excess. Preferably, the conversion is practically complete (starting from a racemate, a maximum conversion of 50% may be reached), and the products are advantageously obtained in good chemical yields and with good enantiomeric excess of at least 70% or 80%, more preferably 90%, still more preferably 95%, most preferably in at least 99% enantiomeric excess.

To further illustrate the present invention, the following is a guide to various terms and wording as used herein, but this is not intended to limit the invention in any way.

A “solvent not miscible with water” as referred to herein is understood to be an organic solvent that shows a reduced solubility in water and, therefore, separates as an upper or lower phase when the concentration of water is increased over its solubility limit. Preferred water non-miscible organic solvents are those having water solubility values (w/w) of less than 50%, more preferably less than 10%, even more preferably less than 1%, and even more preferably less than 0.1%. Non limiting examples of suitable water non-miscible organic solvents include pentyl acetate, tert-pentyl alcohol, anisole, benzene, benzyl alcohol, bromobenzene, 1-butanol, 2-butanol, butyl acetate, butyl ether, chlorobenzene, chloroform, cyclohexane, cyclohexanol, cyclohexanone, cyclopentane, cyclopentyl methyl ether, 1,2-dichlorobenzene, 1,2-dichloroethane, dichloromethane, diethoxymethane, 2-(2-hexylethoxy)ethanol, diisobutyl ketone, dimethoxymethane, ethyl acetate, ethylbenzene, 1,2-diethoxyethane, ethyl ether, n-heptane, n-hexane, 1-hexanol, isoamyl alcohol, isobutanol, isobutyl acetate, isopropyl acetate, isopropyl ether, methyl acetate, methyl tert-butyl ether, methyl cyclohexane, methyl ethyl ketone, methyl formate, methyl isobutyl ketone, 2-methyltetrahydrofuran, nitrobenzene, 1-octanol, n-pentane, 1-pentanol, 3-pentanone, propyl acetate, propylene carbonate, 1-methoxy-2-propanol acetate, propylene oxide, tetrachloroethylene, toluene, 1,1,1-trichloroethane, trichloroethylene, xylene, and mixtures thereof. Particularly preferred water non-miscible organic solvents are selected from the group consisting of 1-butanol, 2-butanol, ethyl acetate, isopropyl acetate, methyl tert-butyl ether, methyl ethyl ketone, methyl isobutyl ketone, toluene, xylene, and mixtures thereof.

The term “protic solvent” as used herein is meant to define any solvent which may contain a dissociable proton suitable for the hydrolysis reaction to occur. Preferably, a protic solvent is a solvent that has a hydrogen atom bound to an oxygen, such as in a hydroxyl group, or to a nitrogen, such as in an amine group. Preferably, the protic solvent of the process of the invention is water.

As referred to herein, (R)-lacosamide as prepared by the invention, and also enantiomeric intermediates useful in the preparation thereof, can be prepared or used in “enantiomerically enriched”, or “enantiomerically pure” form. “Enantiomerically enriched” denotes that the chiral substance, for example (R)-lacosamide or an enantiomeric intermediate useful in the preparation thereof, has an enantiomeric ratio that is greater than 50:50 but less than 100:0. For example, as referred to herein “enantiomerically enriched (R)-lacosamide” comprises an enantiomeric mixture of (R)-lacosamide and (S)-lacosamide which has an enantiomeric excess of said (R)-enantiomer of more than 50%, preferably of at least 70%, preferably of at least 80%, preferably of at least 90%, and preferably of at least 99%. Furthermore, (R)-intermediate (Ma) as herein described, namely (2R)-2-acetylamino-3-methoxypropanoic acid, is a key intermediate useful in the present invention, and is also typically employed in “enantiomerically enriched” form and thereby comprises an enantiomeric mixture of (2R)-2-acetylamino-3-methoxypropanoic acid and (2S)-2-acetylamino-3-methoxypropanoic acid, and having an enantiomeric excess of the (R)-enantiomer of more than 50%, preferably of at least 70%, preferably of at least 80%, preferably of at least 90%, and preferably of at least 99%.

“Enantiomerically enriched” is also used in the context of the present invention to denote in some instances unreacted chiral substance that forms further to the enzymic processes of the present invention, and for example the (S)-enantiomer of a compound of formula (IIa), namely (2S)-2-amino-3-methoxy-N-(phenylmethyl)propanamide as herein described, typically forms as an intermediate mixture with (R)-lacosamide and is present therein in enantiomerically enriched form. Typically, the enantiomerically enriched (2S)-2-amino-3-methoxy-N-(phenylmethyl)propanamide is present as an enantiomeric mixture of (2S)-2-amino-3-methoxy-N-(phenylmethyl)propanamide and (2R)-2-amino-3-methoxy-N-(phenylmethyl)propanamide which has an enantiomeric excess of said (S)-enantiomer of more than 50%, preferably of at least 70%, preferably of at least 80%, preferably of at least 90%, and preferably of at least 99%.

“Enantiomerically pure” as referred to herein typically denotes a sample all of whose molecules have (within limits of detection) the same chirality sense, in other words an (R)-enantiomer substantially free (within limits of detection) of the (S)-enantiomer, for example (R)-lacosamide substantially free (within limits of detection) of (S)-lacosamide.

“Stereoselective hydrolysis or acetylation” as used in the context of the present invention is meant to denote that the enzyme shows a specific selectivity for hydrolysing or acetylating one of the two enantiomers with respect the other. The enzyme preferably shows a selectivity higher than 1%, preferably higher than 25%, preferably higher than 50%, preferably higher than 75%, and preferably higher than 99%, for hydrolysing or acetylating one of the two enantiomers with respect the other.

In the context of the R₁ and/or R₂ definitions of compounds according to the present invention as referred to herein, these can respectively represent “intermediate moieties” that can be converted by known chemical reactions into the desired final moieties of lacosamide, namely CH₃C(═O)—, and —NHCH₂Ph. Examples of suitable moieties are herein described in further detail in the context of processes of the present invention and identification of suitable moieties will be well within the common general knowledge of a person skilled in the art. For example, in the context of R₁, an intermediate moiety can be —H, whereby the resulting amino group can be acetylated to provide the desired acetylamino moiety of lacosamide. For example, in the context of R₂, an intermediate moiety can be —OH, whereby the resulting carboxyl group can be benzylaminated to provide the desired benzylamido moiety of lacosamide.

It will also be appreciated within the context of the broader definitions of processes according to the present invention that enzymic hydrolysis could occur at one or more moieties within the formulae as defined. In the context of the more specific processes as defined herein, however, it will be recognized that certain hydrolysis reactions will be preferred so as to yield (R)-lacosamide as described herein. Specific examples are described herein.

The present invention will now be further illustrated by reference to the following Examples, which do not limit the scope of the invention in any way.

### SPECIFIC EXAMPLES

### General Experimental Conditions

### HPLC Method 1

The chromatographic separation was carried out in a Lux Cellulose-2, 5 μm, 250×4.6 mm I.D chiral column at 40° C. The mobile phase was prepared by mixing n-hexane, ethanol and trifluoroacetic acid (94:6:0.1 v/v/v). The chromatograph was equipped with a 215 nm wavelength detector and the flow rate was 1.0 ml per minute.

### HPLC Method 2

The chromatographic separation was carried out in a Lux Cellulose-2, 5 μm, 4.6 mm×250 mm column at 40° C.

The mobile phase was prepared by mixing isopropanol, ethanol and n-hexane (28:10:62 v/v/v).

The chromatograph was equipped with a 215 nm detector and the flow rate was 0.8 mL/min.

10 μL of the test samples were injected. The test samples were prepared by dissolving the appropriate amount of sample in ethanol, to obtain a concentration of about 2.0 mg per mL, and filtering the resulting solution through a 0.45 μm nylon membrane. The chromatogram was run for at least 45 minutes. Approximate retention time of (R)-lacosamide was 14 minutes. Approximate retention time of (S)-lacosamide was 12 minutes.

### HPLC Method 3

The chromatographic separation was carried out in a Luna C18(2), 5 μm, 4.6 mm×150 mm column at 40° C.

The mobile phase A was a 77:23 (v/v) mixture of buffer (pH 4.0) and methanol. The buffer (pH 4.0) was prepared by dissolving 2.87 g of sodium pentanesulfonate R in 1000 mL of water, and adjusting pH to 4.0 with diluted phosphoric acid R. The mobile phase was mixed and filtered through a 0.22 μm nylon membrane under vacuum.

The mobile phase B was methanol.

The chromatograph was programmed as follows:

Initial 0-7 min. 100% mobile phase A, 7-26 min. linear gradient to 74% mobile phase A, 26-52 min. isocratic 74% mobile phase A, 52-61 min. linear gradient to 100% mobile phase A and 61-70 min. equilibration with 100% mobile phase A.

The chromatograph was equipped with a 217 nm detector and the flow rate was 1.3 mL/min.

10 μL of a reference standard solution of lacosamide were injected. The reference standard solution was prepared by dissolving the appropriate amount of lacosamide in diluent, to obtain a concentration of about 0.0028 mg/mL. The diluent was a 50:50 (v/v) mixture of methanol and water. The chromatogram was run for at least 70 minutes. Approximate retention time of lacosamide was 11 minutes.

10 μL of a reference standard solution of N-benzyl-2-amino-3-methoxypropionamide were injected. The reference standard solution was prepared by dissolving the appropriate amount of N-benzyl-2-amino-3-methoxypropionamide (as oxalate salt) in diluent, to obtain a concentration of about 0.0028 mg/mL (of N-benzyl-2-amino-3-methoxypropionamide oxalate). The diluent was a 50:50 (v/v) mixture of methanol and water. The chromatogram was run for at least 70 minutes. Approximate retention time of N-benzyl-2-amino-3-methoxypropionamide was 18 minutes. The area under the peak obtained for the reference standard solution of N-benzyl-2-amino-3-methoxypropionamide was multiplied by 1.43, which is the ratio between molecular weights of N-benzyl-2-amino-3-methoxypropionamide oxalate and N-benzyl-2-amino-3-methoxypropionamide, to obtain the corrected area under the peak of the reference standard solution of N-benzyl-2-amino-3-methoxypropionamide.

After comparing the area under the peak obtained for the reference standard solution of lacosamide with the corrected area under the peak obtained for the reference standard solution of N-benzyl-2-amino-3-methoxypropionamide, it was concluded that the response factor of N-benzyl-2-amino-3-methoxypropionamide was 1.08 times higher than the response factor of lacosamide.

10 μL of the test samples were injected. The test samples were prepared by dissolving the appropriate amount of sample in diluent, to obtain a concentration of about 2.8 mg/mL, and filtering the resulting solution through a 0.45 μm nylon membrane. The diluent was a 50:50 (v/v) mixture of methanol and water. The chromatogram was run for at least 70 minutes. Peak areas of N-benzyl-2-amino-3-methoxypropionamide were corrected by dividing them by 1.08 (difference in response factors between lacosamide and N-benzyl-2-amino-3-methoxypropionamide).

### Example 1

### Preparation of racemic 2-acetylamino 3-methoxypropanoic acid (compound IIb)

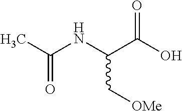

Racemic 2-acetylamino 3-methoxypropanoic acid was prepared starting from DL-serine. First, the amino group was protected with N-tert Butoxycarbonyl, followed by O-methylation of the hydroxylic group, N-deprotection and N-acetylation of the amino group. MS of racemic 2-acetylamino 3-methoxypropanoic acid was 160 uma (ESI, M−1)

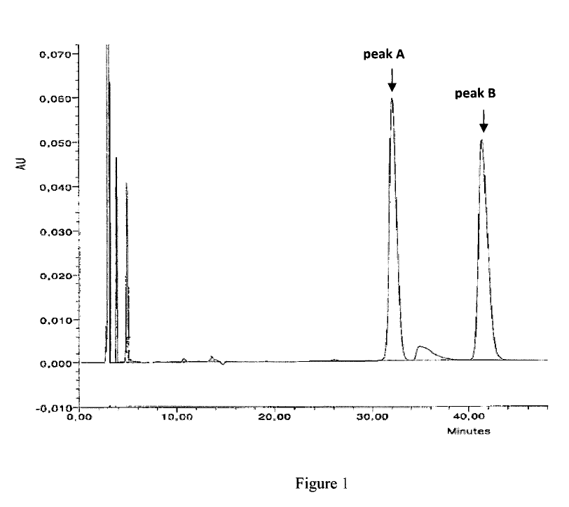

10 μL of a 1% diluted sample of racemic 2-acetylamino 3-methoxypropanoic acid in ethanol was analyzed with the above described chiral HPLC method 1 obtaining the chromatogram depicted in FIG. 1. The chromatogram of FIG. 1 shows two main peaks (peak A and peak B) of equal area which correspond to the two enantiomers of 2-acetylamino 3-methoxypropanoic acid.

### Example 2

### Preparation of (R)-2-acetylamino 3-methoxypropanoic acid (compound IIIa)

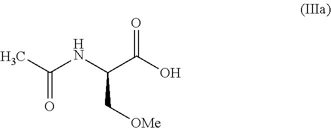

Compound (IIIa) was prepared starting from D-serine. First, the amino group was protected with N-tert butoxycarbonyl, followed by O-methylation of the hydroxylic group, N-deprotection and N-acetylation of the amino group.

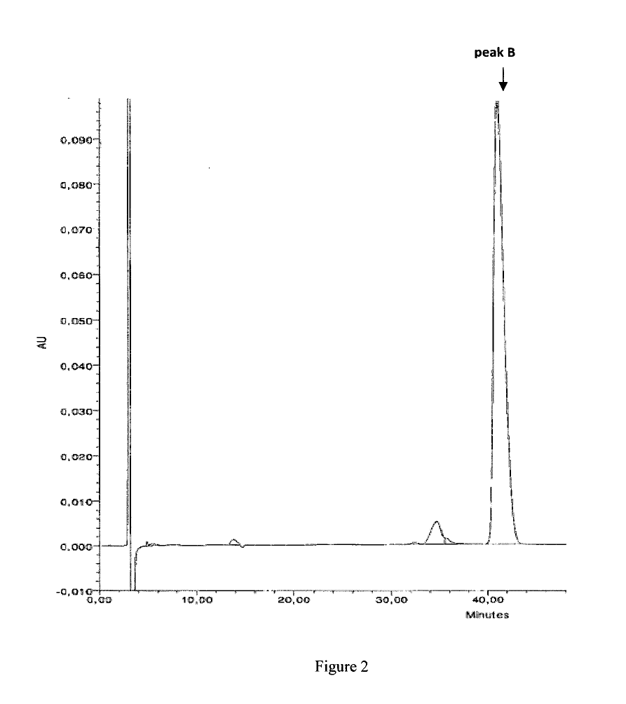

10 μL of a 1% diluted sample of (R)-2-acetylamino 3-methoxypropanoic acid (compound IIIa) in ethanol, was analyzed with the above described HPLC method 1 obtaining the chromatogram depicted in FIG. 2. The chromatogram of FIG. 2 shows one main peak which matches the retention time of peak B of the chromatogram of FIG. 1.

### Example 3

### Preparation of (R)-2-acetylamino 3-methoxypropanoic acid (compound IIIa)

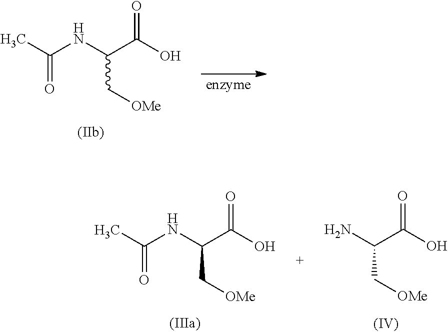

In a 5 ml glass reactor 70 mg of compound (IIb) (0.435 mmol) were placed. Then a solution made of 60 mg (43 units) of Acylase I from Aspergillus melleus (Fluka, cat. No. 01818, activity 0.72 U/mg) and 2 ml of aqueous sodium phosphate buffer at pH 7 was added. The resulting solution was heated to about 37° C.-40° C. and kept for 16 hours with stirring. After that, the mixture was evaporated until dryness and directly analyzed by HPLC.

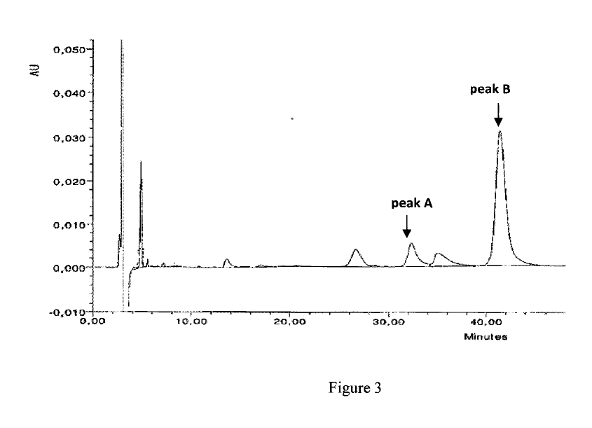

A test sample was prepared as follows: about 100 mg of the reaction mixture crude were weighed, dissolved and diluted to 10 ml with ethanol. 10 μl of this test sample were analyzed with the above described HPLC method 1 obtaining the chromatogram depicted in FIG. 3. The chromatogram of FIG. 3 shows a minor peak which corresponds to peak A (i.e., matches the retention time of peak A of FIG. 1) and a major peak which corresponds to peak B (i.e., matches the retention time of peak B of FIG. 1 and FIG. 2), being the ratio between the peak areas 18.4 (peak B:peak A). According to that result, Acylase I from Aspergillus melleus is able to stereoselectively hydrolyze (deacetylate) the undesired (S) enantiomer of compound (IIb) leaving unreacted the (R) enantiomer of compound (IIIa) as depicted above.

Preparation of pH 7 phosphate buffer: 3.52 g of monobasic potassium phosphate (NaH₂PO₄) and 7.27 g of disodium hydrogen phosphate (Na₂HPO₄) were dissolved in 1000 ml of water and pH was adjusted to about 7.0 with orthophosphoric acid and/or potassium hydroxide.

### Example 4

### Preparation of (R)-N-benzyl-2-acetamido-3-methoxypropionamide [(R)-lacosamide, compound I]

200 mg (0.96 mmol) of racemic N-benzyl-2-amino-3-methoxypropionamide (compound IIa) were dissolved in 10 mL of methyl tert-butyl ether. Then, 0.5 mL (5.11 mmol, 5.3 molar equivalents) of ethyl acetate and 200 mg (1460 units) of Candida antarctica lipase type B (CAL-B, Novozym 435™, 7300 PLU/g) were added to the solution. The resulting suspension was stirred at 22-24° C. for 16 hours. The solvent was removed by evaporation under vacuum. The resulting solid was analyzed by HPLC method 3, and was found to contain 59.7% of lacosamide and 40.3% of unreacted N-benzyl-2-amino-3-methoxypropionamide. The solid was also analyzed by chiral HPLC method 2, showing that the above lacosamide was in form of a mixture of 81% of (R)-lacosamide and 19% of (S)-lacosamide.

PLU/g denotes propyl laurate units per gram. 1 PLU unit=1 μmol of propyl laurate formed per minute and it is a measure of enzyme's activity.

### Example 5

### Preparation of (R)-N-benzyl-2-acetamido-3-methoxypropionamide (lacosamide, compound I)

104.6 mg (0.50 mmol) of racemic N-benzyl-2-amino-3-methoxypropionamide (compound IIa) were dissolved in 8 mL of toluene. Then, 0.4 mL (3.53 mmol, 7.1 molar equivalents) of isopropyl acetate, 20 mg (0.19 mmol, 0.38 molar equivalents) of sodium carbonate, 20 mg (146 units) of Candida antarctica lipase type B (CAL-B, Novozym 435™, 7300 PLU/g) and 21.7 mg (0.020 mmol, 0.04 molar equivalents) of 1-hydroxytetraphenylcyclopentadienyl(tetraphenyl-2,4-cyclopentadien-1-one)-hydrotetracarbonyldiruthenium (II) (Shvo's catalyst) were added to the solution. The resulting mixture was heated to 90° C. and stirred at this temperature for 48 hours. After cooling to room temperature, the mixture was filtered, and the filtrate was evaporated under vacuum. The resulting solid was analyzed by HPLC method 3 and was found to contain a ratio between lacosamide and unreacted N-benzyl-2-amino-3-methoxypropionamide of 93%:7%. The solid was also analyzed by chiral HPLC method 2, showing that lacosamide was in form of a mixture of 86% of (R)-lacosamide and 14% of (S)-lacosamide.

### Example 6

### Preparation of (R)-N-benzyl-2-acetamido-3-methoxypropionamide (lacosamide, compound I)

50 mg (0.24 mmol) of racemic N-benzyl-2-amino-3-methoxypropionamide (compound IIa) were dissolved in 4 mL of toluene. Then, 0.18 mL (1.59 mmol, 6.6 molar equivalents) of isopropyl acetate, 25 mg (0.23 mmol, 0.98 molar equivalents) of sodium carbonate, 0.07 mL (0.48 mmol, 2.00 molar equivalents) of 2,4-dimethylpentan-3-ol, 50 mg (365 units) of Candida antarctica lipase type B (CAL-B, Novozym 435™, 7300 PLU/g) and 21.7 mg (0.025 mmol, 0.1 molar equivalents) of 1-hydroxytetraphenylcyclopentadienyl(tetraphenyl-2,4-cyclopentadien-1-one)-μ-hydrotetracarbonyldiruthenium (II) (Shvo's catalyst) were added to the solution. The resulting mixture was heated to 100° C. and stirred at this temperature for 24 hours. After cooling to room temperature, the mixture was filtered, and the filtrate was evaporated under vacuum. The resulting solid was analyzed by HPLC method 3 and was found to contain a ratio between lacosamide and unreacted N-benzyl-2-amino-3-methoxypropionamide of 99.5%:0.5%. The solid was also analyzed by chiral HPLC method 2, showing that lacosamide was in form of a mixture of 86% of (R)-lacosamide and 14% of (S)-lacosamide.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-40. (canceled)

41. A process of preparing (R)-lacosamide of formula (I): 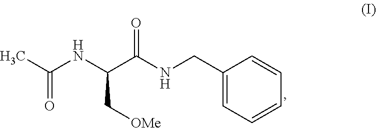 [SMILES: COC[C@@H](NC(C)=O)C(=O)NCc1ccccc1]
comprising:
(i) providing an (R,S)-compound of formula (II): 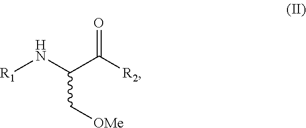
wherein
R₁ is selected from the group consisting of CH₃C(═O)— and a first intermediate moiety capable of being converted into CH₃C(═O)—;
R₂ is selected from the group consisting of —NHCH₂Ph and a second intermediate moiety capable of being converted into —NHCH₂Ph;
(ii) contacting the (R,S)-compound of formula (II) with one or more enzymes in the presence of a solvent and optionally an acetyl donor compound to form an enantiomerically enriched or enantiomerically pure (R)-enantiomer of the compound formula (II),
wherein the one or more enzymes is capable of stereoselectively hydrolyzing either the (R)- or (S)-enantiomer of the compound of formula (II) when the solvent comprises a protic solvent, or is capable of stereoselectively acetylating either the (R)- or (S)-enantiomer of a compound of formula (II) when R₁ is a first intermediate moiety and when an acetyl donor compound is present; and
(iii) optionally further comprising contacting the (R,S)-compound of formula (II) or the enantiomerically enriched or enantiomerically pure (R)-enantiomer of the compound formula (II) with one or more reagents capable of converting a first intermediate moiety into CH₃C(═O)—, and/or said second intermediate moiety into —NHCH₂Ph.

42. The process of claim 41, wherein step (ii) comprises contacting the (R,S)-compound of formula (II) with one or more enzymes in the presence of a solvent and optionally an acetyl donor compound to form an enantiomerically enriched or enantiomerically pure (R)-enantiomer of the compound formula (II) wherein R₁ is CH₃C(═O)—,
wherein the one or more enzymes is capable of stereoselectively hydrolyzing the (R)- or (S)-enantiomer of a compound of formula (II) when the solvent is a protic solvent or is capable of stereoselectively acetylating the (R)-enantiomer of a compound of formula (II) when R₁ is a first intermediate moiety and when an acetyl donor compound is present, and
wherein step (iii) comprises optionally further comprising contacting the (R,S)-compound of formula (II) or the enriched or enantiomerically pure (R)-enantiomer of the compound formula (II) with one or more reagents capable of converting a second intermediate moiety into —NHCH₂Ph.

43. A process of preparing (R)-lacosamide of formula (I): 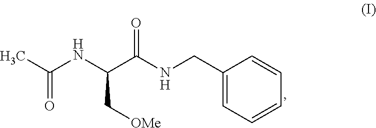 [SMILES: COC[C@@H](NC(C)=O)C(=O)NCc1ccccc1]
comprising:
(i) providing an (R,S)-compound of formula (IIa): 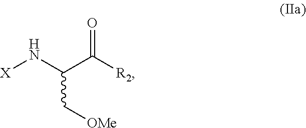
wherein
R₂ is selected from the group consisting of —NHCH₂Ph and an intermediate moiety capable of being converted into —NHCH₂Ph;
X is selected from the group consisting of hydrogen or a leaving group;
(ii) contacting the (R,S)-compound of formula (IIa) with an acetyl donor compound and one or more enzymes capable of stereoselectively acetylating the (R)-enantiomer of a compound of formula (IIa) to form an enantiomerically enriched or enantiomerically pure (R)-enantiomer of the compound formula (III): 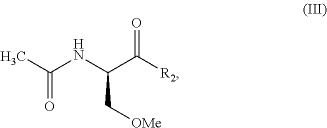
and
(iii) optionally further comprising contacting the (R,S)-compound of formula (IIa) or the enantiomerically enriched or enantiomerically pure (R)-enantiomer of the compound formula (III) with one or more reagents capable of converting an intermediate moiety into —NHCH₂Ph.

44. The process of claim 43, wherein the (R,S)-compound of formula (IIa) is (2R,S)-2-amino-3-methoxy-N(phenylmethyl)propanamide:

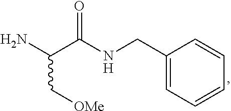 [SMILES: COCC(N)C(=O)NCc1ccccc1]

45. The process of claim 41, wherein the enzyme capable of stereoselectively acetylating either the (R)- or (S)-enantiomer of a compound of formula (II) is a lipase.

46. The process of claim 45, wherein the lipase is selected from the group consisting of Candida antarctica lipase A (CAL-A), Candida antarctica lipase B (CAL-B), and Pseudomonas cepacia lipase.

47. The process of claim 43, wherein the enzyme capable of stereoselectively acetylating the (R)-enantiomer of a compound of formula (IIa) is a lipase.

48. The process of claim 47, wherein the lipase is selected from the group consisting of Candida antarctica lipase A (CAL-A), Candida antarctica lipase B (CAL-B), and Pseudomonas cepacia lipase.

49. The process of claim 41, wherein the acetyl donor compound is selected from the group consisting of acetic acid, acetate esters, acetyl-coenzyme A and acetamides.

50. A process of preparing (R)-lacosamide of formula (I): 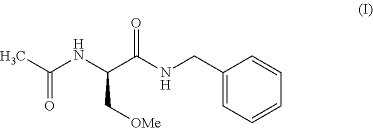 [SMILES: COC[C@@H](NC(C)=O)C(=O)NCc1ccccc1]
comprising:
(i) providing an (R,S)-compound of formula (IIb): 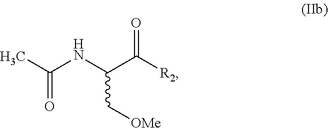
wherein R₂ is selected from the group consisting of —NHCH₂Ph and an intermediate moiety capable of being converted into —NHCH₂Ph;
(ii) contacting the (R,S)-compound of formula (III)) with one or more enzymes capable of stereoselectively hydrolyzing either the (R)- or (S)-enantiomer of the compound of formula (IIb) in the presence of a protic solvent thereby providing an enantiomerically enriched, or enantiomerically pure (R)-enantiomer of a compound of formula (III): 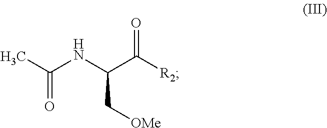
(iii) optionally further comprising contacting an (R,S)-compound of formula (IIb) or the enantiomerically enriched, or enantiomerically pure (R)-enantiomer of a compound of formula (III) with one or more reagents capable of converting the intermediate moiety into —NHCH₂Ph.

51. The process of claim 50, wherein R₂ of the (R,S)-compound of formula (IIb) is —NHCH₂Ph, and wherein the enzyme stereoselectively hydrolyzes the acetylamino group of the (S)-enantiomer of the compound of formula (IIb).

52. The process of claim 50, wherein the (R,S)-compound of formula (IIb) has the formula: 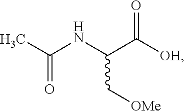
and wherein the enzyme stereoselectively hydrolyzes the acetylamino group of the (S)-enantiomer of the compound of formula (IIb), such that the product of step (ii) is enantiomerically enriched, or enantiomerically pure (R)-enantiomer of a compound of formula (IIIa) and a hydrolysis compound of formula (IV): 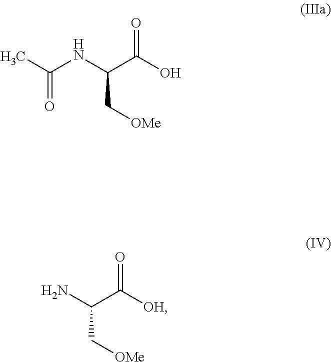
and
wherein step (iii) comprises converting enantiomerically enriched, or enantiomerically pure (R)-enantiomer of a compound of formula (IIIa) into enantiomerically enriched, or enantiomerically pure (R)-lacosamide.

53. The process of claim 41, wherein the enzyme capable of stereoselectively hydrolyzing the (S)-enantiomer of a compound of formula (II) is a hydrolase.

54. The process of claim 53, wherein the hydrolase is Acylase I.

55. The process of claim 50, wherein the one or more enzymes capable of stereoselectively hydrolyzing the (S)-enantiomer of a compound of formula (IIb) is a hydrolase.

56. The process of claim 55, wherein the hydrolase is Acylase I.

57. The process of claim 50, wherein the enzyme stereoselectively hydrolyzes the ester group of the (R)-enantiomer of the compound of formula (IIb) having the formula: 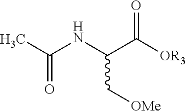
wherein, R₃ is C₁ to C₆ alkyl,
such that the product of step (ii) is enantiomerically enriched, or enantiomerically pure (R)-enantiomer of a compound of formula (IIIa): 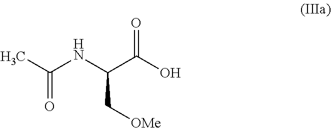
wherein step (iii) comprises converting enantiomerically enriched, or enantiomerically pure (R)-enantiomer of a compound of formula (IIIa) into enantiomerically enriched, or enantiomerically pure (R)-lacosamide.

58. The process of claim 41, wherein the one or more enzymes capable of stereoselectively hydrolyzing the (R)-enantiomer of a compound of formula (II) is selected from the group consisting of a lipase and an esterase.

59. The process of claim 50, wherein the enzyme capable of stereoselectively hydrolyzing the (R)-enantiomer of a compound of formula (IIb) is selected from the group consisting of a lipase or esterase.

60. The process of claim 52, wherein step (iii) comprises contacting the enantiomerically enriched, or enantiomerically pure (R)-enantiomer of a compound of formula (IIIa) with an O-amination activating agent and an O-benzylaminating agent.

61. The process of claim 41, wherein the (R,S)-compound of formula (II) is selected from the group consisting of a racemic mixture or an enantiomerically enriched mixture.

62. The process of claim 43, wherein the (R,S)-compound of formula (IIa) is selected from the group consisting of a racemic mixture or an enantiomerically enriched mixture.

63. The process of claim 43, wherein the acetyl donor compound is selected from the group consisting of acetic acid, acetate esters, acetyl-coenzyme A and acetamides.

64. The process of claim 50, wherein the (R,S)-compound of formula (IIb) is selected from the group consisting of a racemic mixture or an enantiomerically enriched mixture.
